(19) United States
(12) Patent Application Publication
(10) Pub. No.: US 2026/0007421 A1
(43) Pub. Date: Jan. 8, 2026

(54) EXTERNAL HIGH FLOW RATE HEMOSTASIS INLINE VALVE

(71) Applicant: BOSTON SCIENTIFIC SCIMED, INC., Maple Grove, MN (US)

(72) Inventors: Michael William Nagel, Andover, MN (US); Robert D. Cooper, Andover, MN (US); Mark Alfred Hilse, Somerset, WI (US); Michael P. Schrom, Forest Lake, MN (US); Laszlo Trent Farago, Hudson, WI (US)

(73) Assignee: BOSTON SCIENTIFIC SCIMED, INC., Maple Grove, MN (US)

(21) Appl. No.: 19/256,797

(22) Filed: Jul. 1, 2025

Related U.S. Application Data
(60) Provisional application No. 63/666,736, filed on Jul. 2, 2024.

(51) Int. Cl. A61B 17/22; A61B 17/00

(52) U.S. Cl. CPC A61B 17/22; A61B 2017/00137; A61B 2017/00398; A61B 2017/22079

(57) ABSTRACT

Thrombectomy systems including a flow control outflow pump that allows for a free flow of fluid therethrough. An illustrative thrombectomy system may comprise a console, a fluid inflow pump driven by the console, a thrombectomy catheter, the fluid inflow pump configured to provide fluid inflow through the thrombectomy catheter, an effluent waste tube in fluid communication with the thrombectomy catheter, and an outflow pump comprising a stationary portion and a linearly displaceable tube clamp. The outflow pump may be configured to receive the effluent waste tube between the stationary portion and the linearly displaceable tube clamp. The linearly displaceable tube clamp of the outflow pump may be actuatable to selectively compress the effluent waste tube.

### CROSS REFERENCE TO RELATED APPLICATIONS

The present application claims the benefit of U.S. Provisional Patent Application Ser. No. 63/666,736, filed Jul. 2, 2024, the disclosure of which is incorporated herein by reference.

### TECHNICAL FIELD

The disclosure is directed to thrombectomy systems. More particularly, the disclosure is directed to an external high flow rate hemostasis inline valve.

### BACKGROUND

Thrombectomy is a procedure for removing thrombus from the vasculature of a patient. Mechanical and fluid-based systems can be used to remove thrombus. With fluid-based systems, an infusion fluid may be infused to a treatment area of a vessel with a catheter to dislodge the thrombus. In some instances, an effluent (e.g., the infusion fluid and/or blood) including the dislodged thrombus may be extracted from the vessel through the catheter. Of the known thrombectomy systems and methods, there is an ongoing need to provide alternative configurations of thrombectomy catheters and systems, as well as methods of operating such thrombectomy systems.

### SUMMARY

This disclosure provides design, material, manufacturing method, and use alternatives for medical devices.

In a first example, a thrombectomy system may comprise a console, a fluid inflow pump, the fluid inflow pump driven by the console, a thrombectomy catheter, the fluid inflow pump configured to provide fluid inflow through the thrombectomy catheter, an effluent waste tube in fluid communication with the thrombectomy catheter, and an outflow pump comprising a stationary portion and a linearly displaceable tube clamp, the outflow pump configured to receive the effluent waste tube between the stationary portion and the linearly displaceable tube clamp. The linearly displaceable tube clamp of the outflow pump may be actuatable to selectively compress the effluent waste tube.

Alternatively or additionally to any of the examples above, in another example, the linearly displaceable tube clamp may be movable towards the stationary portion to compress the effluent waste tube to block a flow of fluid therethrough.

Alternatively or additionally to any of the examples above, in another example, the linearly displaceable tube clamp may be movable away from the stationary portion to expand a cross-sectional area of the effluent waste tube to allow a flow of fluid therethrough.

Alternatively or additionally to any of the examples above, in another example, the linearly displaceable tube clamp may be actuatable to increase or decrease a flow rate of fluid through the effluent waste tube.

Alternatively or additionally to any of the examples above, in another example, in a first configuration, the linearly displaceable tube clamp may compress the effluent waste tube to block a flow of fluid therethrough.

Alternatively or additionally to any of the examples above, in another example, in a second configuration, the linearly displaceable tube clamp may be spaced a distance from the effluent waste tube.

Alternatively or additionally to any of the examples above, in another example, in the second configuration, a flow of fluid through the effluent waste tube may be unobstructed.

Alternatively or additionally to any of the examples above, in another example, when the fluid inflow pump is active, the linearly displaceable tube clamp may be positioned to allow a flow of fluid through the effluent waste tube.

Alternatively or additionally to any of the examples above, in another example, when the fluid inflow pump is inactive, the linearly displaceable tube clamp may be positioned to block a flow of fluid through the effluent waste tube.

Alternatively or additionally to any of the examples above, in another example, during an upstroke of the fluid inflow pump, the linearly displaceable tube clamp may be positioned to block a flow of fluid through the effluent waste tube.

Alternatively or additionally to any of the examples above, in another example, during a downstroke of the fluid inflow pump, the linearly displaceable tube clamp may be positioned to allow a fluid flow through the effluent waste tube.

Alternatively or additionally to any of the examples above, in another example, a position of the linearly displaceable tube clamp may be based, at least in part on an expected outflow flow rate.

Alternatively or additionally to any of the examples above, in another example, the expected outflow flow rate may be stored within a data plate on the thrombectomy catheter or the fluid inflow pump.

Alternatively or additionally to any of the examples above, in another example, the thrombectomy system may further comprise a linear actuator movably coupled relative to the linearly displaceable tube clamp.

Alternatively or additionally to any of the examples above, in another example, an actuator arm of the linear actuator may be configured to extend to move the linearly displaceable tube clamp towards the stationary portion and to retract to move the linearly displaceable tube clamp away from the stationary portion.

In another example, a thrombectomy system may comprise a console, a fluid inflow pump, the fluid inflow pump driven by the console, a thrombectomy catheter, the fluid inflow pump configured to provide fluid inflow through the thrombectomy catheter, an effluent waste tube in fluid communication with the thrombectomy catheter, an outflow pump comprising a stationary portion and a linearly displaceable portion, the outflow pump configured to receive the effluent waste tube between the stationary portion and the linearly displaceable portion, a linear actuator comprising a housing and an actuator arm, and a pivot arm extending between the actuator arm and the linearly displaceable portion of the outflow pump. The linear actuator may be configured to laterally displace the linearly displaceable portion of the outflow pump to selectively allow a flow of fluid through the effluent waste tube.

Alternatively or additionally to any of the examples above, in another example, when the fluid inflow pump is active, the linearly displaceable portion of the outflow pump may be positioned to allow the flow of fluid through the effluent waste tube.

Alternatively or additionally to any of the examples above, in another example, when the fluid inflow pump is inactive, the linearly displaceable portion of the outflow pump may be positioned to block the flow of fluid through the effluent waste tube.

Alternatively or additionally to any of the examples above, in another example, the linearly displaceable portion may be movable towards the stationary portion to compress the effluent waste tube. In another example, a console for a thrombectomy system may comprise a carriage assembly disposed within an interior of the console, the carriage assembly configured to receive a pump/catheter assembly, a reciprocating linear actuator configured to drive a pump of the pump/catheter assembly, and an outflow pump comprising a stationary portion and a linearly displaceable portion, the outflow pump configured to receive an effluent waste tube between the stationary portion and the linearly displaceable portion. The linearly displaceable portion of the outflow pump may be actuatable to selectively compress the effluent waste tube to vary a flow rate of fluid within the effluent waste tube.

The above summary of some example embodiments is not intended to describe each disclosed embodiment or every implementation of the disclosure.

### BRIEF DESCRIPTION OF THE DRAWINGS

The disclosure may be more completely understood in consideration of the following detailed description of various embodiments in connection with the accompanying drawings, in which:

FIG. 1 is a perspective view of an illustrative thrombectomy system;

FIG. 2 is a partially exploded perspective view of several components of an illustrative pump/catheter assembly;

FIG. 3 is a perspective view of an illustrative outflow pump assembly;

FIG. 4 is a top view of the illustrative outflow pump assembly of FIG. 3 in a first configuration; and

FIG. 5 is a top view of the illustrative outflow pump of FIG. 3 in a second configuration.

While the disclosure is amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

All numeric values are herein assumed to be modified by the term “about”, whether or not explicitly indicated. The term “about” generally refers to a range of numbers that one of skill in the art would consider equivalent to the recited value (e.g., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

Thrombectomy catheters and systems may be used to remove thrombus, plaques, lesions, clots, etc. from veins or arteries. In some thrombectomy systems, a roller pump or an outflow pump may be used to balance fluid flow in the system. For example, a roller pump may be used to control the flow of effluent from a patient through the system while an inflow pump provides a flow of fluid into the patient. However, in some instances, a roller pump may not operate fast enough to provide the necessary fluid balance between the fluid inflow and the fluid outflow (e.g., effluent). For example, the high-pressure inflow fluid may cause the rapid influx of blood into the thrombectomy catheter at a high flow rate. In some cases, more fluid may be drawn into the thrombectomy catheter than can exit (e.g., Qᵢₙ>Qₒᵤₜ of the thrombectomy catheter). This imbalance in fluid flow may cause fluid regurgitation which may lead to hemolysis and/or distal embolization. In some cases, the limitation of fluid outflow may be limited by a pump within the thrombectomy system. To prevent hemolysis and/or distal embolization, it is contemplated that the outflow of the thrombectomy catheter (e.g., Qₒᵤₜ) should exceed or be equal to the inflow into the thrombectomy catheter (e.g., Qᵢₙ). Disclosed herein are thrombectomy catheters and systems that may improve the functionality of the thrombectomy catheter and/or system.

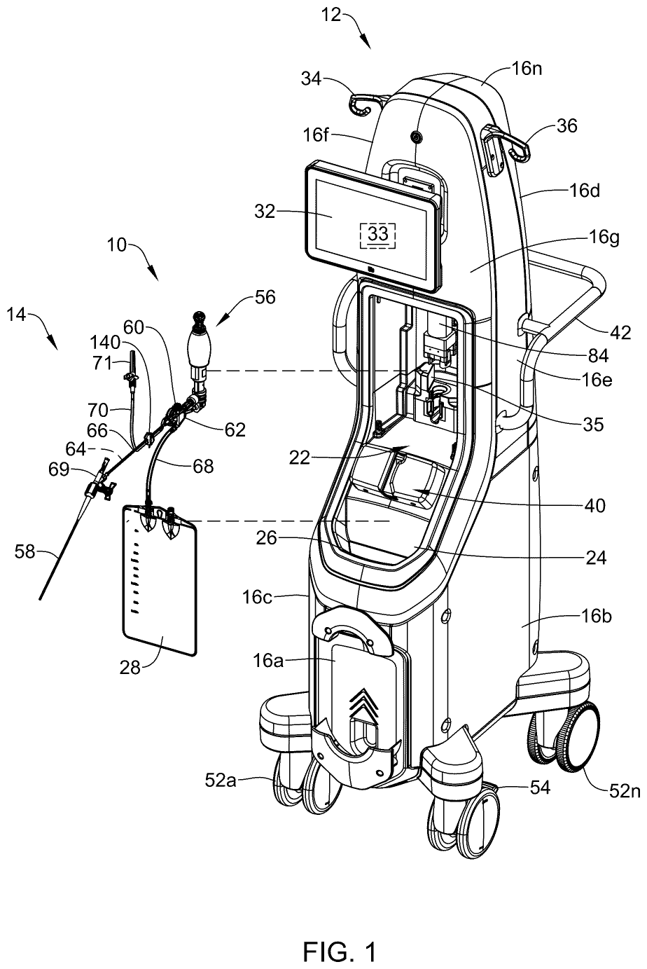

FIG. 1 is a perspective view of an illustrative thrombectomy system 10. The thrombectomy system 10 may include a control console 12, including a drive unit, and a pump/catheter assembly 14. In some instances, the pump/catheter assembly 14 may be a disposable single use device in which a new pump/catheter assembly 14 may be used with the console 12 for each medical procedure. The console 12 may include a housing enclosing the internal structure of the console 12. Shown on the console 12 are a plurality of removable panels 16a-16n about and along the console 12 enclosing the internal structure of the console 12. An illustrative console 12 is described in commonly assigned U.S. Pat. No. 7,935,077, titled THROMBECTOMY CATHETER DEPLOYMENT SYSTEM, the disclosure of which is hereby incorporated by reference. Centrally located in the console 12 and aligned to the lower region of the panel 16g may be automatically opening loading bay door assemblies (not explicitly shown) which open to expose the interior of the console 12 to provide access to a carriage assembly 22 and close during use of the pump/catheter assembly 14. Illustrative loading bay doors and assemblies are described in commonly assigned U.S. patent application Ser. No. 18/604,956, titled THROMBECTOMY SYSTEM WITH LOADING BAY DOORS, the disclosure of which is hereby incorporated by reference.

The console 12 may include a catch basin or drip tray 24 for collecting fluid leakage from the components of the pump/catheter assembly 14. In some instances, the drip tray 24 may be removable. Other configurations of catch basins are also contemplated. The drip tray 24 and/or a receptacle 26 may collectively support and accommodate an effluent collection bag, such as effluent collection bag 28 of the pump/catheter assembly 14. In other instances, the console 12 may include a different structure, such as a hook for hanging the effluent collection bag 28 from, or a shelf for setting the effluent collection bag 28 on. The effluent waste tube 68 may also be positioned in an outflow or roller pump 40 between the tube guides with the effluent collection bag 28 connected to the effluent waste tube 68. The effluent collection bag 28 may be suitably positioned for collecting effluent during the medical procedure. Pump rollers (not shown) of the roller pump 40 may rotatably engage the effluent waste tube 68 to control effluent fluid flow through the effluent waste tube 68 to the effluent collection bag 28.

In instances where the carriage assembly 22 is movable, a carriage assembly activation switch (not explicitly shown) may be provided with the console 12, such as located on a panel 16g, to selectively position the carriage assembly 22 inwardly or outwardly. In other instances, the carriage assembly 22 may be positioned or moved using a control panel and/or user interface 32. A user interface 32, including memory and/or processing capabilities, may be provided with the console 12, such as located at the upper region of the console 12 between the upper regions of the upper side panels 16e and 16f. The user interface 32 may be a guided user interface (GUI) including a touch screen display to allow a user to provide input to the user interface 32 and view information on a same display screen. However, this is not required. In other instances, the user input may be separate from the display screen.

Saline bag hooks 34 and 36 may extend through the panels 16e and 16f to hang saline bags therefrom. The console 12 may include a handle 42 as well as a plurality of wheels 52a-52n and brake pedals 54 for wheel lockage to assist in maneuvering the console 12 by medical personnel.

In FIG. 1, the pump/catheter assembly 14 is shown detached from the console 12. The pump/catheter assembly 14 includes an inflow pump 56 and a thrombectomy device 58. In some embodiments, the inflow pump 56 may be configured to provide fluid inflow through the thrombectomy device 58. During use, a portion of the pump/catheter assembly 14 may be secured within a portion of the console 12. In some embodiments, the pump/catheter assembly 14 may include a bubble trap 60 attached to the inflow pump 56, a connection manifold assembly 62 connected to the bubble trap 60, a fixture 140, an effluent return tube 66 connected between the connection manifold assembly 62 and the thrombectomy device 58, a high-pressure fluid supply tube 64 attached between the output of the inflow pump 56 and the thrombectomy device 58 which may be coaxially arranged inside the effluent return tube 66, a transition fixture 69 between the distal end of the effluent return tube 66 and the proximal end of the thrombectomy device 58, an effluent waste tube 68 connecting the effluent collection bag 28 to the connection manifold assembly 62, and a fluid supply tube 70 having a bag spike 71 connecting a fluid supply bag (e.g., a saline bag) (not explicitly shown) to the connection manifold assembly 62. The fluid supply tube 70 may be in fluid communication with the interior of the bubble trap 60 to provide fluid from the fluid supply bag to the inflow pump 56 and then to the thrombectomy device 58 through the high-pressure fluid supply tube 64.

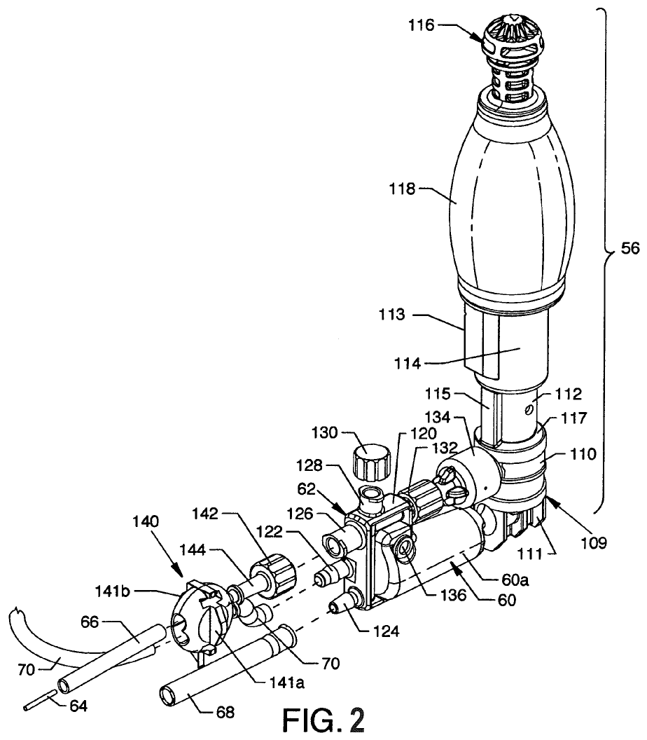

The console 12 may include a reciprocating linear actuator 84 configured to engage a pump piston head 116 (see, for example, FIG. 2) of the inflow pump 56 when the inflow pump 56 is engaged with the carriage assembly 22. The reciprocating linear actuator 84 may be disposed within the interior of the drive unit 12 and may be aligned with the pump piston head 116 (e.g., FIG. 2) when the inflow pump 56 is disposed within the interior of the drive unit 12. The reciprocating linear actuator 84 may be actuated such that 20) reciprocating (e.g., up and down) strokes of the reciprocating linear actuator 84 drive the inflow pump 56 in response to activation of a user activation switch (not explicitly shown) or a control command from a controller 33. In some embodiments, the user activation switch may be a foot switch. In some embodiments, when the user activation switch is depressed, the reciprocating linear actuator 84 and/or the inflow pump 56 is activated 25 and/or runs, and when the user activation switch is released, the reciprocating linear actuator 84 and/or the inflow pump 56 is stopped and/or ceases operation.

The console 12 may include a controller 33 in electronic communication with the user interface 32, the reciprocating linear actuator 84, the inflow pump 56, the roller pump 40, and/or the thrombectomy device 58. In some cases, the controller 33 may be a part of or otherwise incorporated into the user interface 32. In some embodiments, the console 12 and/or the controller 33 may include a data acquisition device 35. In some embodiments, the data acquisition device 35 may be disposed within the interior of the drive unit 12. For example, the data acquisition device 35 may be positioned on or near the carriage assembly 22. In some embodiments, the data acquisition device 35 may be configured for wireless communication. Other configurations are also contemplated. The data acquisition device 35 may be a radiofrequency identification reader and/or a barcode reader. Other types of data acquisition devices 35 may be used, as desired. In some embodiments, the user activation switch may be in electronic communication with the drive unit 12 and/or the controller 33. In some embodiments, the user activation switch may be in electronic communication with the drive unit 12 and/or the controller 33 via a wire or cable. In some embodiments, the user activation switch may be in electronic communication with the drive unit 12 and/or the controller 33 wirelessly.

FIG. 2 is a partially exploded perspective view of several components of the pump/catheter assembly 14 (e.g., FIG. 1) generally including the inflow pump 56, the bubble trap 60, the connection manifold assembly 62, and the fixture 140. The inflow pump 56 centers about a tubular body 112. Components are located about the lower region of the tubular body 112 and include a base 109 having an upper portion 110 and a lower portion 111 both positioned about the lower region of the tubular body 112. An annular surface 117 is included at the top of the upper portion 110 of the base 109 for intimate contact with capture tabs of the carriage assembly 22 (e.g., FIG. 1) to retain the inflow pump 56 within and/or in engagement with the carriage assembly 22. A top body 114, is positioned about the upper region of the tubular body 112. The base 109 and the top body 114, as well as a connecting panel 115, may be molded or otherwise suitably constructed to encompass the greater part of the tubular body 112, for example.

In some embodiments, a data plate 113 may also be included on the pump/catheter assembly 14, such as on the top body 114 for example, for the inclusion of a barcode, an radiofrequency identification (RFID) tag, a data storage chip, informational displays, etc. to store, communicate, and/or otherwise determine specifications and/or operational parameters associated with the pump/catheter assembly 14, the thrombectomy device 58, the inflow pump 56, etc. and/or components thereof. In at least some embodiments, the data acquisition device 35 (e.g., FIG. 1) may be configured to communicate with the data plate 113 (e.g., the barcode, the RFID tag, the data storage chip, etc.) to obtain specifications, operational parameters, and/or identifying information associated with the pump/catheter assembly 14, the thrombectomy device 58, the inflow pump 56, etc., and/or components thereof.

In some embodiments, the inflow pump 56 may include a hemispherically-shaped pump piston head 116 having a flexible boot 118 connected to and extending between the top body 114 and the pump piston head 116. In some instances, the lower portion 111 of the base 109 may serve as a mount for a first end of the bubble trap 60.

The connection manifold assembly 62 may be secured directly to a second end of the bubble trap 60 and in some instances may include a bracket 120 to which is attached a vertically oriented tubular manifold 148 having a plurality of ports attached or formed therethrough including a fluid (e.g., saline) inlet port 122, an effluent outlet port 124, a Luer style effluent return port 126, and/or an auxiliary port 128 and cap 130. Also shown are connectors 132 and 134 connectingly extending between the connection manifold assembly 62 and the upper portion 110 of the base 109.

The bubble trap 60 may include two mating halves of which a first bubble trap half 60a is shown. A hydrophobic filter 136 may be included at the upper forward region of the first bubble trap half 60a. In some embodiments, a second hydrophobic filter may be included on the second bubble trap half (not explicitly shown) which opposes the hydrophobic filter 136 on the first bubble trap half 60a.

The fixture 140, and components associated therewith, assists in support and connection of the effluent return tube 66 to the effluent return port 126 by a connector 142 combined continuously with a connection tube 144, and also assists in support, passage, and connection of the fluid supply tube 70 with the fluid inlet port 122. The fixture 140 may include outwardly extending vertically aligned and opposed tabs 141a and 141b which prevent the fixture 140 and associated effluent return tube 66 containing the high-pressure fluid supply tube 64 and the fluid supply tube 70 from contacting the roller pump 40 (e.g., FIG. 1) provided with the drive unit 12, such as located in the carriage assembly 22 or adjacent thereto.

In some embodiments, the effluent waste tube 68 may be positioned within and/or through the outflow pump with the effluent collection bag 28 (e.g., FIG. 1) connected to the effluent waste tube 68. The effluent collection bag 28 may be suitably positioned for collecting effluent during the procedure. In some embodiments, pump rollers of the roller pump 40 may engage the effluent waste tube 68 to control effluent fluid flow through the effluent waste tube 68 from the effluent outlet port 124 to the effluent collection bag 28. In other embodiments, the pump rollers of the roller pump 40 may be disengaged from the effluent waste tube 68 to allow for free flow of the effluent. In some embodiments, other pump types and/or configurations may be used in place of the roller pump.

At an appropriate time, the thrombectomy device 58 may be subjected to a priming procedure to purge the thrombectomy device 58 of any air. For example, the tip of the thrombectomy device 58 may be placed in a bowl of sterile saline, or other fluid, and the inflow pump 56 may be operated by action of the reciprocating linear actuator 84 (such as by activating and/or depressing the user activation switch) to prime the thrombectomy device 58. Thereafter, medical personnel may insert the thrombectomy device 58 into the vasculature of the patient, and operation of the thrombectomy system 10 incorporating the user interface 32 and the user activation switch can begin, as desired. The reciprocating linear actuator 84 is actuated according to the operating parameters to influence fluid inflow pressures, pump speed, flow rates, and the like to operate the inflow pump 56 to deliver pressurized fluid to the thrombectomy device 58 via the high-pressure fluid supply tube 64 residing in the effluent return tube 66. Supply fluid is routed through the bubble trap 60, may be pressurized by the inflow pump 56, and is routed through the high-pressure fluid supply tube 64 to the thrombectomy device 58 for use in a thrombectomy or other related procedure. Effluent may be returned through the effluent return tube 66 to the connection manifold assembly 62 for collection in the effluent collection bag 28 (e.g., FIG. 1) through the effluent waste tube 68.

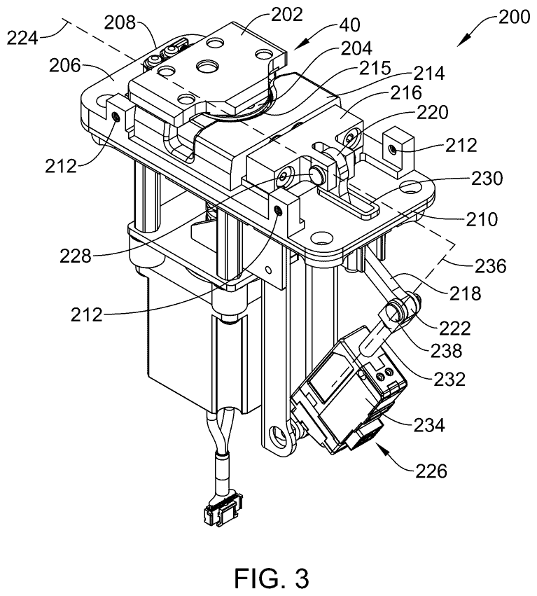

As describe herein, in some cases, the outflow pump 40 may not operate fast enough to provide the necessary fluid balance between the fluid inflow and the fluid outflow (e.g., effluent). For example, the fluid inflow may exceed the fluid outflow flow rate. FIG. 3 is a perspective view of an illustrative outflow pump assembly 200 including the outflow pump 40 that may be used to allow for free flow of effluent when the reciprocating linear actuator 84 is operating and to prevent effluent flow when the reciprocating linear actuator 84 is not operating. In FIG. 3, an upper cover of the outflow pump 40 has been removed to more particularly illustrate the inner components of the outflow pump 40. Generally, the outflow pump 40 may include a stationary portion and a linearly displaceable portion. The linearly displaceable portion may be movable to facilitate insertion of the effluent waste tube 68 between the stationary portion and the linearly displaceable portion. Further, the linearly displaceable portion may be movable between a number of different positions to compress the effluent waste tube 68 to reduce a flow of fluid therethrough to varying extents or to allow the effluent waste tube 68 to be free from a compressive force.

The outflow pump 40 may include a top cap 202 disposed over and enclosing a roller frame 204 and one or more rollers (not explicitly shown). The top cap 202 may be secured to a pump base 206. The pump base 206 may extend from a first end 208 to a second end 210. The pump base 206 may include features such as, but not limited to, one or more threaded apertures 212, configured to be used to secure the outflow pump assembly 200 to an upper cover and/or to the console 12.

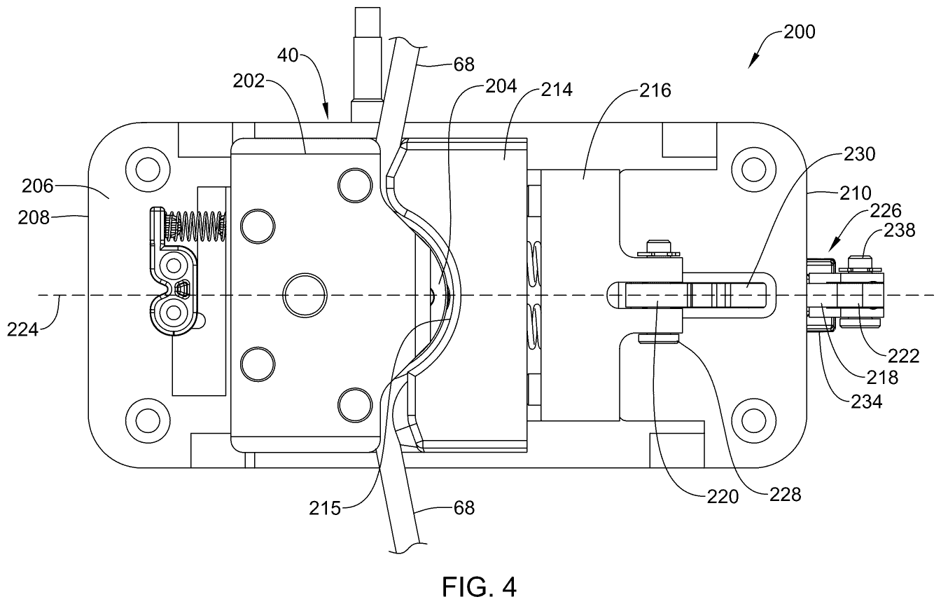

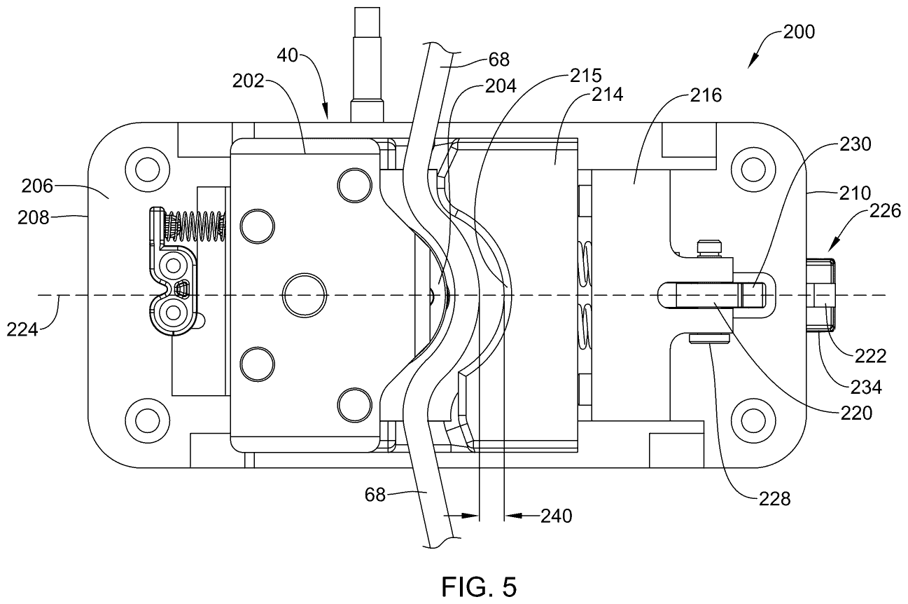

The outflow pump 40 may further include a tube clamp 214. The tube clamp 214 may be actuatable between a plurality of positions to selectively position a fluid tube (e.g., the effluent waste tube 68) (see, for example, FIGS. 4 and 5) in contact between a clamping surface 215 of the tube clamp 214 and at least one of the one or more rollers while the top cap 202 and the roller frame 204 remain stationary. The clamping surface 215 may be oriented or facing towards the top cap 202 and the roller frame 204. In some cases, the roller frame 204, the one or more rollers, and/or other mechanisms to control rotation of the pump rollers may be omitted. For example, a stationary post or protrusion may replace one or more of the at least one roller such that the effluent waste tube 68 is positioned between the post and the clamping surface 215 of the tube clamp 214. It is contemplated that the tube clamp 214 may be used to compress the fluid tube. It is contemplated that compressing the fluid tube may reduce the inner diameter or cross-sectional area thereof and thus reduce the flow of fluid therethrough. Thus, the flow rate of the fluid through the effluent waste tube 68 may be varied by varying a position of the tube clamp 214 to change an inner diameter or cross-sectional area of the effluent waste tube 68. For example, reduction of the cross-sectional area of the effluent waste tube 68 may reduce a flow rate of fluid therethrough. When the tube clamp 214 is in a position closest to the roller frame 204, the flow of fluid through the effluent waste tube 68 may be the most restricted or the slowest. In some cases, the flow of fluid through the effluent waste tube 68 may be substantially or completely stopped or blocked. Moving the tube clamp 214 in a direction away from the roller frame 204 may allow the inner diameter or cross-sectional area of the effluent waste tube 68 to expand and the flow rate to increase. The tube clamp 214 may be incrementally movable between a first configuration closest to the roller frame 204 or the first end 208 of the pump base 206 and a second configuration laterally spaced from the roller frame 204 relative to the first configuration and/or closer to the second end 210 of the pump base 206.

The tube clamp 214 may be secured to a platen pusher block 216. Collectively, the tube clamp 214 and the platen pusher block 216 may form a linearly displaceable portion of the outflow pump 40. However, in some cases one of the tube clamp 214 or the platen pusher block 216 may be omitted. The platen pusher block 216 may be coupled to a pivot arm 218. Rotational movement of the pivot arm 218 may be translated to linear movement of the platen pusher block 216 and the tube clamp 214. For example, the pivot arm 218 may include a first end 220 pivotably coupled to the platen pusher block 216 via a pin 228 such that rotation of the pivot arm 218 moves the platen pusher block 216 and the tube clamp 214 in a direction generally parallel to a longitudinal axis 224 of the pump base 206. For example, the pin 228 may extend through one or more apertures (not explicitly shown) of the platen pusher block 216 and an aperture extending through the first end 220 of the pivot arm 218. The pivot arm 218 may extend from a second end 222 pivotably coupled to an electric linear actuator 226, through a slot or opening 230 in the pump base 206 to the first end 220 coupled to the platen pusher block 216. In some embodiments, the platen pusher block 216 may be omitted and the pivot arm may be connected directly to the tube clamp 214.

The linear actuator 226 may include an actuator arm 232 and a housing 234. The second end 222 of the pivot arm 218 may be pivotably coupled to a free end of the actuator arm 232 via a pin 238. The actuator arm 232 may be actuatable along a longitudinal axis 236 thereof. In the illustrated embodiment, the actuator arm 232 is shown in an extended configuration. In the illustrated embodiment, when the actuator arm 232 is in the extended configuration, the tube clamp 214 and the platen pusher block 216 are positioned in a first configuration adjacent to the roller frame 204 which compresses the effluent waste tube 68 to the greatest extent. However, this is not required. In some cases, the tube clamp 214 and the platen pusher block 216 are positioned in a second configuration spaced a distance from the roller frame 204 and are free from contact with the effluent waste tube 68 when the actuator arm 232 is in the extended configuration. The actuator arm 232 may be retracted into the housing 234 of the linear actuator 226. As the actuator arm 232 is retracted into the housing 234 of the linear actuator 226, the actuator arm 232 pulls the second end 222 of the pivot arm and translates linear motion of the actuator arm 232 to rotational movement of the pivot arm 218. The torque is transferred to the platen pusher block 216 and the tube clamp 214 from the first end 220 of the pivot arm 218 to draw the tube clamp 214 and the 216 linearly along the slot 230. It is contemplated that the actuator arm 232 may be positioned anywhere between a fully extended configuration (shown in FIG. 3) and a fully retracted configuration (not explicitly shown) to position the platen pusher block 216 and the tube clamp 214 anywhere between the first configuration which restricts a flow of fluid to the greatest degree and the second configuration which allows for free flow of fluid through the effluent waste tube 68 (e.g., there is no compressive force exerted on the effluent waste tube 68). For example, the linear actuator 226 may include a rotary encoder which allows for precise positioning of the actuator arm 232 and thus the tube clamp 214 and the platen pusher block 216. The linear actuation of the actuator arm 232 may be reversed to extend the actuator arm 232 and move the tube clamp 214 and the platen pusher 20) block 216 from the second, fully open, configuration to the first most flow restricted configuration. It some cases, the first configuration substantially or completely prevents a flow of fluid through the effluent waste tube 68.

FIG. 4 is a top view of the illustrative pump assembly 200 in the first configuration and FIG. 5 is a top view of the illustrative pump assembly 200 in the second configuration. As described herein, in some cases, the outflow pump 40 may not be able to run fast enough to achieve fluid balance (e.g., the effluent outflow rate attainable by the outflow pump 40 may be significantly less than the fluid inflow generated by the pump 56 such that a desired fluid balance between the fluid inflow and the effluent outflow is not able to be achieved). In such a situation, an alternative configuration of the effluent pathway may be provided, as described herein. For example, a flow of the fluid through the effluent pathway may adjusted using a position of portions of the outflow pump 40. An illustrative method for controlling a flow of outflow using the pump assembly 200 will be described with respect to FIGS. 4 and 5.

In some cases, the outflow pump 40 may function as an on/off mechanism to selectively allow a flow of fluid (e.g., outflow) through the effluent waste tube 68. As will be described in more detail herein, the outflow pump 40 is positioned in line with the effluent waste tube 68 and is configured to allow flow of effluent through the effluent waste tube 68 to the waste bag 28 when the pump/catheter assembly 14 is in use (i.e., when the pump 56 is activated and running to provide fluid inflow through the thrombectomy device 58) and prevent a flow of effluent through the effluent waste tube 68 to the waste bag 28 when the pump/catheter assembly 14 is not in use (i.e., when the pump 56 is deactivated and not running). To prevent fluid flow through the effluent waste tube 68, the tube clamp 214 may be advanced towards the roller frame 204 to position the effluent waste tube 68 between at least one roller and the clamping surface 215 of the tube clamp 214. The tube clamp 214 may be advanced towards the roller frame 204 to position the tube clamp 214 in the first configuration with the actuator arm 232 fully extended, as shown in FIG. 4. Said differently, in the first configuration, the tube clamp 214 is positioned as close to the roller frame 204 as structurally possible. In the first configuration, the clamping surface 215 of the tube clamp 214 may apply a biasing or pushing force to the effluent waste tube 68 to push the effluent waste tube 68 against at least one roller to collapse or pinch the walls of the effluent waste tube 68 together to block or substantially block a flow of fluid through the effluent waste tube 68.

When the pump/catheter assembly 14 is in use, the actuator arm 232 may be retracted into the housing 234 to move the tube clamp 214 away from the roller frame 204 and towards the second configuration, as shown in FIG. 5. In the second configuration, the tube clamp 214 is laterally displaced from the roller frame 204 in a direction towards the second end 210 of the pump base 206 to position the clamping surface 215 of the tube clamp 214 a distance 240 (FIG. 5) from the effluent waste tube 68. The distance 240 may be determined by one or more of a length of the slot 230, a length of the pivot arm 218, and/or a length of the actuator arm 232. In the second configuration, the distance 240 between the effluent waste tube 68 and the clamping surface 215 of the tube clamp 214 is at a maximum. It is contemplated that the tube clamp 214 need not be moved entirely to the second configuration to release the compressive force on the effluent waste tube 68.

It is contemplated that the linear actuator 226 may be controlled in a number of different manners. In some embodiments, control of the linear actuator 226 may be linked to the pump 56. For example, when the pump 56 is activated, the actuator arm 232 of the linear actuator 226 is retracted to laterally displace the tube clamp 214 to allow unobstructed flow of fluid through the effluent waste tube 68. When the pump 56 is deactivated, the actuator arm 232 of the linear actuator 226 is extended to laterally displace the tube clamp 214 to obstruct or block a flow of fluid through the effluent waste tube 68.

It is contemplated that the linear actuator 226 or the controller 33 of the console 12 may be programmed such that the linear actuator 226 is in a normally closed state (e.g., the actuator arm 232 extended to position the tube clamp 214 to contact and exert a biasing force on the effluent waste tube 68) when the pump/catheter assembly 14 is not in use and actuated to an open configuration when the pump/catheter assembly 14 is activated (e.g., the actuator arm 232 is retracted to move the tube clamp 214 away from the effluent waste tube 68.

In other examples, control of the linear actuator 226 may correspond to movement of the reciprocating linear actuator 84. For example, during upstroke of the reciprocating linear actuator 84 (and the pump 56) entrainment is not observed and clot removal is not occurring. However, blood loss still occurs. To minimize blood loss, the linear actuator 226 may be configured to be in phase with the reciprocating linear actuator 84. For example, during the upstroke of the reciprocating linear actuator 84, the actuator arm 232 of the linear actuator 226 may be extended to block or partially block the effluent waste tube 68 with a biasing force from the tube clamp 214. During the downstroke of the reciprocating linear actuator 84, the actuator arm 232 of the linear actuator 226 may be retracted to move the tube clamp 214 away from the effluent waste tube 68 to allow for more flow through the effluent waste tube 68.

In any of the control mechanisms described herein, it is contemplated that the tube clamp 214 does not necessarily need to be positioned a maximum distance 240 from the effluent waste tube 68. For example, the positioning of the tube clamp 214 may be based, at least in part, on an expected outflow flow rate. For example, if the flow rate of the outflow is not expected to exceed the flow rate of the inflow, the tube clamp 214 may be positioned to at least partially reduce an inner diameter or cross-sectional area of the effluent waste tube 68 (relative to an unbiased configuration) to throttle the flow rate of the outflow to match the flow rate of the inflow. However, if the flow rate of the outflow in a throttled configuration (e.g., a reduced inner diameter or cross-sectional area of the effluent waste tube 68 or using the outflow pump 40 to drive fluid flow) is expected to exceed the flow rate of the inflow, the tube clamp 214 may be positioned such that there is no reduction in the inner diameter of the effluent waste tube 68. In some cases, the controller 33 or the linear actuator 226 may receive flow rate information particular to the device being used via information stored within the data plate 113 of the pump/catheter assembly 14. Alternatively, or additionally, the flow rate of the outflow may be measured with a flow sensor. The measured flow rate may then be used to control a position of the tube clamp 214.

The materials that can be used for the various components of the thrombectomy catheter, pump/catheter assembly, and/or other devices disclosed herein may include those commonly associated with medical devices. For simplicity purposes, the following discussion makes reference to the pump/catheter assembly and its related components. However, this is not intended to limit the devices and methods described herein, as the discussion may be applied to other similar devices, tubular members and/or components of tubular members or devices disclosed herein.

The various components of the devices/systems disclosed herein may include a metal, metal alloy, polymer (some examples of which are disclosed herein), a metal-polymer composite, ceramics, combinations thereof, and the like, or other suitable material. Some examples of suitable metals and metal alloys include stainless steel, such as 304V, 304 L, and 316 LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-NR and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly (alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly (styrene-b-isobutylene-b-styrene) (for example, SIBS and/or SIBS A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In at least some embodiments, portions or all of the pump/catheter assembly and its related components may be doped with, made of, or otherwise include a radiopaque material. Radiopaque materials are understood to be materials capable of producing a relatively bright image on a fluoroscopy screen or another imaging technique during a medical procedure. This relatively bright image aids the user of the pump/catheter assembly and its related components in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the pump/catheter assembly and its related components to achieve the same result.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A thrombectomy system, comprising:
a console;
a fluid inflow pump, the fluid inflow pump driven by the console;
a thrombectomy catheter, the fluid inflow pump configured to provide fluid inflow through the thrombectomy catheter;
an effluent waste tube in fluid communication with the thrombectomy catheter; and
an outflow pump comprising a stationary portion and a linearly displaceable tube clamp, the outflow pump configured to receive the effluent waste tube between the stationary portion and the linearly displaceable tube clamp;
wherein the linearly displaceable tube clamp of the outflow pump is actuatable to selectively compress the effluent waste tube.

2. The thrombectomy system of claim 1, wherein the linearly displaceable tube clamp is movable towards the stationary portion to compress the effluent waste tube to block a flow of fluid therethrough.

3. The thrombectomy system of claim 1, wherein the linearly displaceable tube clamp is movable away from the stationary portion to expand a cross-sectional area of the effluent waste tube to allow a flow of fluid therethrough.

4. The thrombectomy system of claim 1, wherein the linearly displaceable tube clamp is actuatable to increase or decrease a flow rate of fluid through the effluent waste tube.

5. The thrombectomy system of claim 1, wherein in a first configuration, the linearly displaceable tube clamp compresses the effluent waste tube to block a flow of fluid therethrough.

6. The thrombectomy system of claim 5, wherein in a second configuration, the linearly displaceable tube clamp is spaced a distance from the effluent waste tube.

7. The thrombectomy system of claim 6, wherein in the second configuration, a flow of fluid through the effluent waste tube is unobstructed.

8. The thrombectomy system of claim 1, wherein when the fluid inflow pump is active, the linearly displaceable tube clamp is positioned to allow a flow of fluid through the effluent waste tube.

9. The thrombectomy system of claim 1, wherein when the fluid inflow pump is inactive, the linearly displaceable tube clamp is positioned to block a flow of fluid through the effluent waste tube.

10. The thrombectomy system of claim 1, wherein during an upstroke of the fluid inflow pump, the linearly displaceable tube clamp is positioned to block a flow of fluid through the effluent waste tube.

11. The thrombectomy system of claim 1, wherein during a downstroke of the fluid inflow pump, the linearly displaceable tube clamp is positioned to allow a fluid flow through the effluent waste tube.

12. The thrombectomy system of claim 1, wherein a position of the linearly displaceable tube clamp is based, at least in part on an expected outflow flow rate.

13. The thrombectomy system of claim 12, wherein the expected outflow flow rate is stored within a data plate on the thrombectomy catheter or the fluid inflow pump.

14. The thrombectomy system of claim 1, further comprising a linear actuator movably coupled relative to the linearly displaceable tube clamp.

15. The thrombectomy system of claim 14, wherein an actuator arm of the linear actuator is configured to extend to move the linearly displaceable tube clamp towards the stationary portion and to retract to move the linearly displaceable tube clamp away from the stationary portion.

16. A thrombectomy system, comprising:
a console;
a fluid inflow pump, the fluid inflow pump driven by the console;
a thrombectomy catheter, the fluid inflow pump configured to provide fluid inflow through the thrombectomy catheter;
an effluent waste tube in fluid communication with the thrombectomy catheter;
an outflow pump comprising a stationary portion and a linearly displaceable portion, the outflow pump configured to receive the effluent waste tube between the stationary portion and the linearly displaceable portion;
a linear actuator comprising a housing and an actuator arm; and
a pivot arm extending between the actuator arm and the linearly displaceable portion of the outflow pump;
wherein the linear actuator is configured to laterally displace the linearly displaceable portion of the outflow pump to selectively allow a flow of fluid through the effluent waste tube.

17. The thrombectomy system of claim 16, wherein when the fluid inflow pump is active, the linearly displaceable portion of the outflow pump is positioned to allow the flow of fluid through the effluent waste tube.

18. The thrombectomy system of claim 16, wherein when the fluid inflow pump is inactive, the linearly displaceable portion of the outflow pump is positioned to block the flow of fluid through the effluent waste tube.

19. The thrombectomy system of claim 16, wherein the linearly displaceable portion is movable towards the stationary portion to compress the effluent waste tube.

20. A console for a thrombectomy system, the console comprising:
a carriage assembly disposed within an interior of the console, the carriage assembly configured to receive a pump/catheter assembly;
a reciprocating linear actuator configured to drive a pump of the pump/catheter assembly; and
an outflow pump comprising a stationary portion and a linearly displaceable portion, the outflow pump configured to receive an effluent waste tube between the stationary portion and the linearly displaceable portion;
wherein the linearly displaceable portion of the outflow pump is actuatable to selectively compress the effluent waste tube to vary a flow rate of fluid within the effluent waste tube.
